(19) United States
(12) Patent Application Publication
(10) Pub. No.: US 2025/0375169 A1
(43) Pub. Date: Dec. 11, 2025

(54) MOTION ACTIVITY BASED ESCALATION OF HEART RATE SENSOR

(71) Applicant: Apple Inc., Cupertino, CA (US)

(72) Inventors: Neda Ehtiati, San Diego, CA (US); Mariah W. Whitmore, Cupertino, CA (US); Richard A. Fineman, Campbell, CA (US); William R. Powers, III, San Francisco, CA (US)

(21) Appl. No.: 19/231,341

(22) Filed: Jun. 6, 2025

Related U.S. Application Data
(60) Provisional application No. 63/657,706, filed on Jun. 7, 2024.

(51) Int. Cl. A61B 5/00; A61B 5/024; A61B 5/11

(52) U.S. Cl. CPC A61B 5/7285; A61B 5/02438; A61B 5/1123

(57) ABSTRACT

Embodiments of a framework are disclosed for stair escalation to capture high resolution heart rate data that can be used to develop cardio fitness metrics related to stair climbing.

### CROSS-REFERENCE TO RELATED APPLICATION

This application claims priority to U.S. Provisional Patent Application No. 63/657,706, filed Jun. 7, 2024, then entire contents of which are incorporated herein by reference.

### TECHNICAL FIELD

This disclosure relates generally to health and fitness monitoring.

### BACKGROUND

Wearables such as a smartwatch can provide various metrics indicating a user's cardio fitness such as V02max estimation, heart rate recovery, etc. In general, these metrics have better availability or accuracy if the user participates in fitness activities with high exertion such as exercise sessions.

Climbing stairs is one of the daily activities that historically has been used for fitness assessment for patients with cardiovascular disease symptoms. Part of the initial questionnaire for such patients is to assess their difficulty climbing 1 or 2 flight of stairs. Therefore, deriving a cardio fitness metric post climbing stairs based on sensor data provided by wearables, such as a smartphone or smartwatch, can be beneficial in assessing the user's cardio fitness level.

To achieve metrics evaluating the user cardio fitness during stair climbing, it is desirable to have access to heart rate (HR) data with high sampling rate. One of the limiting factors in wearable devices is battery life and power usage especially for sensors with high power demand. For example, in some smart watches, the satellite navigation system (e.g., Global Positioning System (GPS)), inertial sensors (e.g., gyroscope) and high-resolution HR sensor are examples of sensors that are activated on demand to save battery life.

Accordingly, for health monitoring and fitness applications that need data from these sensors, additional logic is needed to activate these sensors opportunistically based on the user's activity. Opportunistic events to collect sensor data are referred to herein as “escalations.”

### SUMMARY

Embodiments of a framework are disclosed for stair escalation to capture high resolution HR data that can be used to develop cardio fitness metrics related to stair climbing.

In some embodiments, a method comprises: obtaining a window of climbing probability samples, the climbing probability samples indicating the probability that a user of the wearable device is climbing stairs; computing a stair metric based on the window of climbing probability samples; validating the stair metric; obtaining a window of vertical speed samples, the vertical speed samples indicating the vertical speed of the wearable device; determining an ascending or descending condition based on the window of vertical speed samples; and initiating escalation of a heart rate sensor based on the validated stair metric and the ascending or descending condition.

In some embodiments, a system comprises: at least one processor; memory storing instructions, that when executed by the at least one processor, cause the at least one processor to perform operations comprising: obtaining a window of climbing probability samples, the climbing probability samples indicating the probability that a user of the wearable device is climbing stairs; computing a stair metric based on the window of climbing probability samples; validating the stair metric; obtaining a window of vertical speed samples, the vertical speed samples indicating the vertical speed of the wearable device; determining an ascending or descending condition based on the window of vertical speed samples; and initiating escalation of a heart rate sensor based on the validated stair metric and the ascending or descending condition.

Particular embodiments described herein provide one or more of the following advantages. The disclosed framework implements escalation during stair climbing by activating an HR sensor earlier than existing algorithms to capture, e.g., peak HR data. The more accurate HR data can be used to derive more accurate cardio fitness metrics post stair climbing which can be more beneficial in assessing the user's cardio fitness level than provided by existing algorithms

### BRIEF DESCRIPTION OF THE DRAWINGS

FIG. 1 is a plot illustrating a latency problem with existing stair detection and stair speed algorithms, according to one or more embodiments.

FIG. 2 is a flow diagram of a process of validating the detection of stairs, according to one or more embodiments.

FIG. 3 is a flow diagram of a process of detecting ascending or descending stairs, according to one or more embodiments.

FIG. 4 is a state machine for adapting HR recovery for true and false events, according to one or more embodiments.

FIG. 5 is a flow diagram of a process of motion activity-based escalation of HR sensor, according to one or more embodiments.

FIG. 6 is a block diagram of a wearable device architecture for implementing the features and processes described in reference to FIGS. 1-5, according to some embodiments.

### DETAILED DESCRIPTION

### Overview

HR achieved post stair climbing is a strong indicator of cardiorespiratory decline. For example, the ability to climb zero to two flights of stairs is used to grade the severity of angina in a patient (chest pain due to reduced blood flow to the heart). Some key cardio metrics for stair climbing include HR recovery after climb and top of stairs HR. Thus, it is desirable to activate the HR sensor on a wearable device early enough during stair climbing to capture peak HR.

There are multiple existing methods that detect stairs on a smartwatch (described below). These include, for example, a flight detection algorithm which computes increments of flights ascended or descended based on pedometer data, and a stair speed algorithm that provides an estimation of stair speed up or down based on changes in barometric pressure data provided by a barometric sensor and angular rate data provided by a gyroscope sensor.

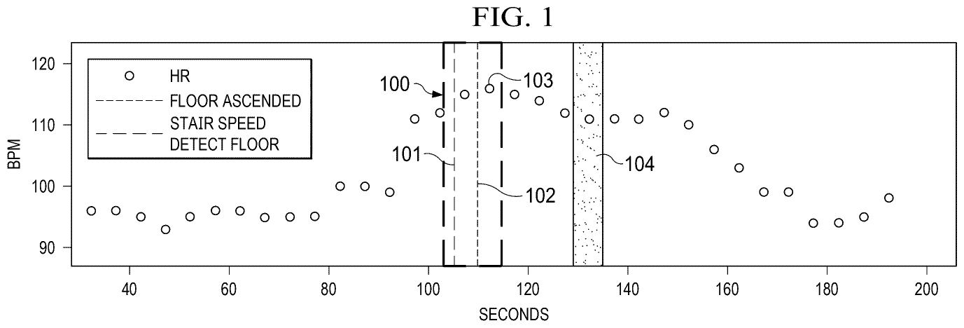

FIG. 1 is a plot illustrating a latency problem with existing stair detection and stair speed algorithms, according to one or more embodiments. A plot of heart rate in beats per minute (bpm) versus time is shown. In a climbing event window 100, a user climbs a flight of stairs. A stair detector and stair speed detector worn by the user detects a floor at time 101 and the user reaches the top of stairs at time 102. At time 104, the algorithms finished their respective processing and the heart rate sensor is activated.

As shown in FIG. 1 the latency to detect the leading flight of stairs in the climbing event window 100 using a flight detection and stair speed algorithm is longer than the time 102 needed to climb one flight of stairs, i.e., the leading flight is detected after climbing it. There is also an additional latency due to communication of HR data between hardware components and HR sensor warm up time. The HR sensor escalation logic based on the two existing algorithms has accumulated latency which prevents these algorithms to activate the HR sensor promptly to capture the user's elevated heart rate post climbing stairs. As shown in FIG. 1, the user's elevated HR post climbing stairs has settled substantially by the time the HR sensor is activated. Thus, the peak elevated HR 103 is not captured because the HR sensor was activated too late due to the latency.

To address the latency issue, the disclosed method uses intermediate values to determine the probability of climbing stairs and also the direction of climb (ascending versus descending). The disclosed embodiments, in general, detect the stairs earlier than the existing algorithms and therefore can be used to promptly turn on the HR sensor to capture the elevated heart rate post stair climbing.

The stair escalation logic is based on the following two metrics: Valid Stair metric and Asc/Desc metric. The Valid Stair metric functions as a voting mechanism on climbing probability (provided by the stair detection algorithm) to improve the reliability of a stair speed metric and limit false detections. The Asc/Desc metric is a moving sum of the sign of vertical speed in the range that corresponds to climbing stairs. These two metrics are used in a state machine to initiate stair escalation. The state machine limits the duration of false positive escalations that can have a negative impact on the power consumption of the wearable device. The false escalations can be caused by, for example, erroneous values for the climbing probability which is calculated based on a pressure filtered signal which can have erroneous values due to pressure differences between rooms or water ingress.

### Example Stair Detection Validation

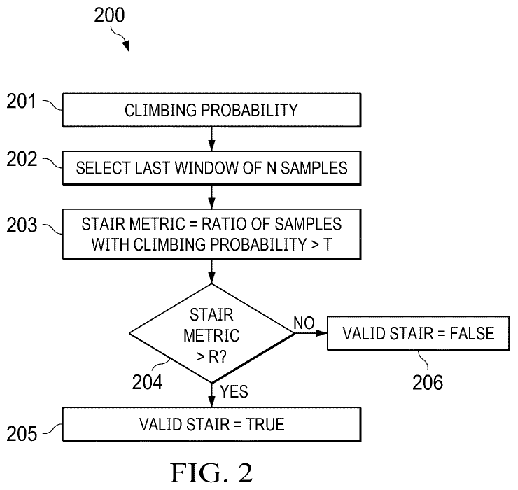

FIG. 2 is a flow diagram of a process 200 of validating the detection of stairs, according to one or more embodiments. Process 200 begins by obtaining consecutive windows of N samples of climbing probability from a stair speed algorithm (201).

In an embodiment, a stair speed algorithm determines how quickly the user. is climbing up a flight of continuous stairs regardless of the geometry of the stairs, such as multi flight stairs with landings in between flights of stairs. A flight detection algorithm (described below) can be used to determine when the user starts to climb a flight of stairs. A change of pressure (e.g., provided by a barometer sensor) is then monitored until the pressure becomes constant which indicates the user has reached the top of the stairs, i.e., no more change in elevation. In some embodiments, the change in height over the time between the user starting to climb and reaching the top of the stairs gives the stair speed. In embodiments with multiple flights of stairs with landings in between, the landings are detected (e.g., based on detecting a constant average vertical speed/oscillation) and removed from the stair speed calculation.

Referring again to FIG. 2, process 200 continues by selecting the last window of N samples (202) and computing a stair metric (203) as, e.g., a ratio of vertical displacement over the step count in the last window of N samples that is above a threshold T. If (204) the stair metric is greater than a threshold R, Valid Stair=TRUE (205); otherwise, Valid Stair=FALSE (206).

### Example Process for Detecting Ascending Descending Stairs

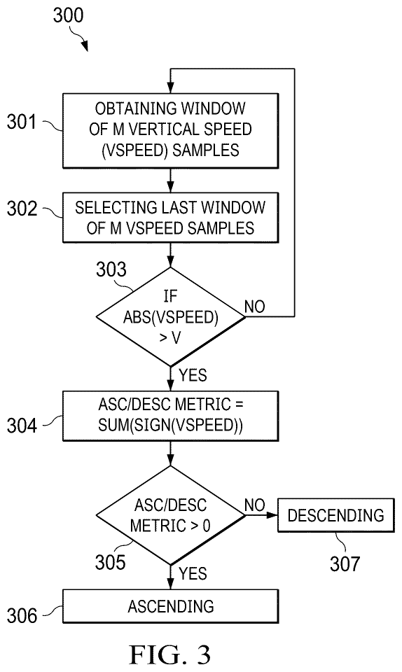

FIG. 3 is a flow diagram of a process 300 of detecting ascending or descending stairs, according to one or more embodiments. Process 300 begins by obtaining a window of M samples of the vertical speed (vspeed) of the wearable device (301). In some embodiments, the vertical speed samples can be obtained from acceleration data output by accelerometers embedded in the wearable device. In some embodiments, an estimated vertical speed can be obtained from an estimation filter, such as a Kalman Filter which accounts for process and measurement noise.

Process 300 continues by selecting a last window of M samples of vertical speed (302) and determining if the absolute value of the vertical speed is greater than a vertical speed threshold V (303). If the absolute value of the vertical speed is greater than the vertical speed threshold V, the Asc/Desc metric is set equal to the moving sum of the sign of the vertical speed calculated in the given window of M samples (304), and if the Asc/Desc metric is greater than zero (305), the stairs are ascending (306); otherwise, the stairs are descending (307). If (303) the absolute value of the vertical speed is not greater than the vertical speed threshold V, process 300 returns to step 301.

### Example State Machine for Initiating Escalation

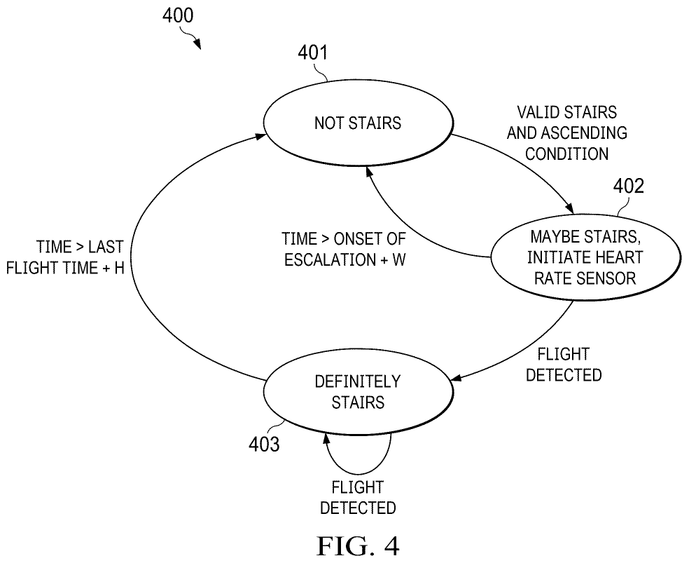

FIG. 4 is a state machine 400 for adapting HR recovery for true and false positive events, according to one or more embodiments. The two metrics Valid Stair and Asc/Desc determined in processes 200, 300 are used in the state machine 400 to initiate stair escalation. The goal for the state machine 400 is to limit the duration of false positive escalations that can have negative impact on the power consumption of the wearable device. The false positive escalations can be caused by, for example, erroneous values for the climbing probability which is calculated based on a pressure filtered signal which can have erroneous values due to the pressure difference between rooms or water ingress.

In some embodiments, the state machine 400 has 3 states: Not Stairs (401), Maybe Stairs (402), Definitely Stairs (403). The state transitions occur based on the following logic. The transition from Not Stairs to Maybe Stairs occurs if Valid Stair=TRUE and the Asc/Desc metric indicates an ascending condition. This transition initiates stair escalation and turns on the HR sensor.

The transition from Maybe Stairs to Not Stairs occurs if within a certain time window (length W) after the onset of escalation (Time>onset of escalation+W), a flight is not detected based on a flight detection algorithm, where W is the first flight wait time defined as the time between when the Valid Stair metric indicates stair and the flight detector indicates floor. This transition indicates a false positive and the escalation is terminated.

In some embodiments, the flight detection algorithm is implemented as a classifier (e.g., support vector machine (SVM), classification tree, deep learning network). Some examples of features that are input into the flight detection classifier include but are not limited to: a rate of change of pressure (e.g., provided by a barometer sensor), a step count provided by a digital pedometer (e.g., based on a pendulum model), time domain acceleration data (e.g. to obtain the periodicity of the acceleration signal indicative of a climbing cadence) and frequency domain acceleration data and/or pressure data. For example, the power in different frequency bands (e.g., between 1-4 Hz typical walking frequency) for acceleration and/or pressure can be features input into the flight detection classifier. In some embodiments, the vertical displacement divided by the number of steps taken over a window of time, i.e., vertical displacement per step, is a feature input into the flight detection classifier. Vertical displacements can be computed from pressure change data provided by a pressure sensor (e.g., a barometer).

In some embodiments, the time domain acceleration data is integrated to obtain vertical speed (to determine vertical displacement of the user), which is then fused with the pressure data (also a measure of vertical displacement) to detect a change in vertical displacement of the user with each stair step taken by the user, where a regular spacing in the vertical displacement is indicative of the user climbing a stair.

In some embodiments, location data is used to increase the probability of stairs when the user is in physical proximity to an area known to contain stairs from previous observations and/or map data.

In some embodiments, the output of the stair detection classifier is a class or probability. For example, if the output of the stair detection classifier is 0.8, then there is high probability that the user is climbing stairs. By contrast, if the output of the stair detection classifier is 0.3, then there is a low probability that the user is climbing stairs. The stair detection algorithm can be trained based on a database of pressure change, step count and energy profile training data using techniques known in the art.

If visual data is available (e.g., images from a camera), then the stair detection classifier can be, e.g., a convolutional neural network (e.g., faster R-CNN) that is trained on images of different types of staircases (e.g., straight, curved, spiral, L-shaped, U-shaped, winder, multiple flight stairs with landings between each flight) captured under various conditions (e.g., lighting conditions). The detection classifier can take images of stairs captured by a camera of a wearable device as input and predict when the user is ascending or descending stairs. In some embodiments, one or more classifiers can be trained to detect stair ascension/descension based on a combination of inertial sensor and visual data.

Referring again to FIG. 4, the transition from Maybe Stairs to Definitely Stairs occurs if within a certain time window (length W) after the onset of escalation a flight is detected by the flight detection algorithm, where W is the first flight wait time defined as the time between when the Valid Stair metric indicates stair and the flight detector indicates floor. In this state, the escalation is extended while a continuous flight of stairs with time intervals less than a given time delta is detected.

The transition from Definitely Stairs to Not Stairs occurs after the Last Flight Time, where the escalation is extended by a constant time (length H) (Time>Last Flight Time+H), where H is the stair recovery time defined as the time to extend escalation after the last floor is detected to capture HR recovery. In this state, the escalation is turned off.

### Parameter Optimization Methodology

Using the flight detection algorithm to be an early indicator of stair climbing after onset of escalation enables the turn off of the escalation in a short amount of time, limiting the duration of false positives and the effect on the power budget of the wearable device. In some instances, the imperfections in the pressure sensor data used by the flight detection algorithm is causing an elevated number of false positives which can consume the allowable power budget for escalations quickly. Adding the additional metrics described above and defining state machine 300 are mechanisms to improve the accuracy of the stair escalations. However, the stair speed and flight detection algorithms include design parameters that can be optimized based on a design requirement. These design parameters can directly affect the accuracy of the disclosed stair escalation algorithm.

To optimize the design parameters, the connection between design requirements such as power budget limitation and data availability and the number of false positives and true positives is used. The design parameters are optimized to achieve the maximum true positive rate while limiting the false positive rate.

In some embodiments, subjective parameter settings can be assumed, where the previous history of stair climbing for the user or other behavioral/daily routines or demographic indicators, e.g., weight, height, age and body mass index (BMI) are used to tune the design parameters. In some embodiments, other sensor streams from the wearable device can be included in the current model to improve the accuracy of the stair detection.

### Example Process of Motion Activity Based Escalation of HR Sensor

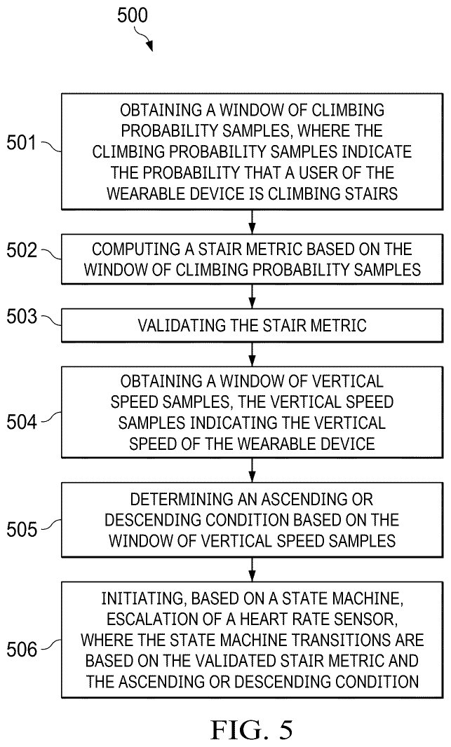

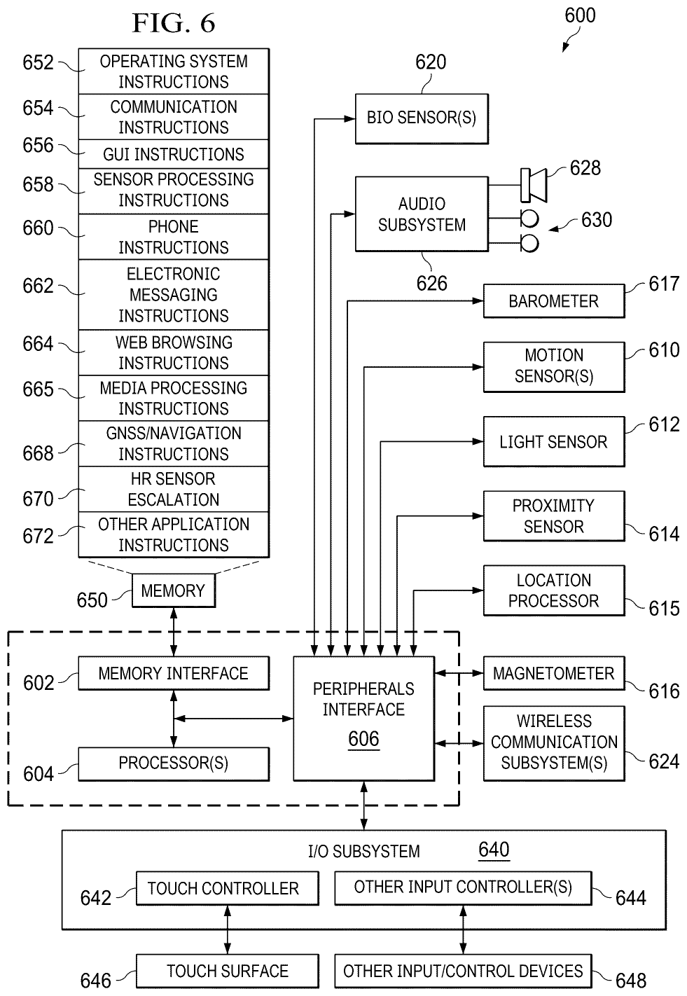

FIG. 5 is a flow diagram of process 500 of motion activity based escalation of a HR sensor, according to one or more embodiments. Process 500 can be implemented using, for example, the wearable device architecture 600 described in reference to FIG. 6.

Process 500 begins by obtaining a window of climbing probability samples, where the climbing probability samples indicate the probability that a user of the wearable device is climbing stairs (501).

Process 500 continues by computing a stair metric based on the window of climbing probability samples (502) and validating the stair metric (503).

Process 500 continues by obtaining a window of vertical speed samples, the vertical speed samples indicating the vertical speed of the wearable device (504).

Process 500 continues by determining an ascending or descending condition based on the window of vertical speed samples (505).

Process 500 continues by initiating, based on a state machine, escalation of a heart rate sensor, where the state machine transitions are based on the validated stair metric and the ascending or descending condition (506).

Each of the steps of process 500 was previously described in reference to FIGS. 1-4.

### Example Device Architecture

FIG. 6 is a block diagram of a wearable device architecture 600 for implementing the features and processes described in reference to FIGS. 1-5. Architecture 600 can include memory interface 602, one or more hardware data processors, image processors and/or processors 604 and peripherals interface 606. Memory interface 602, one or more processors 604 and/or peripherals interface 606 can be separate components or can be integrated in one or more integrated circuits. System architecture 600 can be included in any suitable electronic device, including but not limited to: a smartwatch, smartphone, fitness band and any other device that can be attached, worn or held by a user.

Sensors, devices and subsystems can be coupled to peripherals interface 606 to provide multiple functionalities. For example, one or more motion sensors 610, light sensor 612 and proximity sensor 614 can be coupled to peripherals interface 606 to facilitate motion sensing (e.g., acceleration, rotation rates), lighting and proximity functions of the wearable device. Location processor 615 can be connected to peripherals interface 606 to provide geo-positioning. In some implementations, location processor 615 can be a GNSS receiver, such as the Global Positioning System (GPS) receiver. Electronic magnetometer 616 (e.g., an integrated circuit chip) can also be connected to peripherals interface 606 to provide data that can be used to determine the direction of magnetic North. Electronic magnetometer 616 can provide data to an electronic compass application. Motion sensor(s) 610 can include one or more accelerometers and/or gyros configured to determine change of speed and direction of movement. Barometer 617 can be configured to measure atmospheric pressure. Bio signal sensor 620 can be one or more of a HR sensor (e.g., a PPG sensor), an electroencephalogram (EEG) sensor, an electrocardiogram (ECG) sensor, an electromyogram (EMG) sensor, a mechanomyogram (MMG) sensor (e.g., piezo resistive sensor) for measuring muscle activity/contractions, an electrooculography (EOG) sensor, a galvanic skin response (GSR) sensor, a magnetoencephalogram (MEG) sensor and/or other suitable sensor(s) configured to measure bio signals.

Communication functions can be facilitated through wireless communication subsystems 624, which can include radio frequency (RF) receivers and transmitters (or transceivers) and/or optical (e.g., infrared) receivers and transmitters. The specific design and implementation of the communication subsystem 624 can depend on the communication network(s) over which a mobile device is intended to operate. For example, architecture 600 can include communication subsystems 624 designed to operate over a GSM network, a GPRS network, an EDGE network, a Wi-Fi™ network and a Bluetooth™ network. In particular, the wireless communication subsystems 624 can include hosting protocols, such that the mobile device can be configured as a base station for other wireless devices.

Audio subsystem 626 can be coupled to a speaker 628 and a microphone 630 to facilitate voice-enabled functions, such as voice recognition, voice replication, digital recording and telephony functions. Audio subsystem 626 can be configured to receive voice commands from the user.

I/O subsystem 640 can include touch surface controller 642 and/or other input controller(s) 644. Touch surface controller 642 can be coupled to a touch surface 646. Touch surface 646 and touch surface controller 642 can, for example, detect contact and movement or break thereof using any of a plurality of touch sensitivity technologies, including but not limited to capacitive, resistive, infrared and surface acoustic wave technologies, as well as other proximity sensor arrays or other elements for determining one or more points of contact with touch surface 646. Touch surface 646 can include, for example, a touch screen or the digital crown of a smart watch. I/O subsystem 640 can include a haptic engine or device for providing haptic feedback (e.g., vibration) in response to commands from processor 604. In an embodiment, touch surface 646 can be a pressure-sensitive surface.

Other input controller(s) 644 can be coupled to other input/control devices 648, such as one or more buttons, rocker switches, thumbwheel, infrared port and USB port. The one or more buttons (not shown) can include an up/down button for volume control of speaker 628 and/or microphone 630. Touch surface 646 or other controllers 644 (e.g., a button) can include, or be coupled to, fingerprint identification circuitry for use with a fingerprint authentication application to authenticate a user based on their fingerprint(s).

In one implementation, a pressing of the button for a first duration may disengage a lock of the touch surface 646; and a pressing of the button for a second duration that is longer than the first duration may turn power to the mobile device on or off. The user may be able to customize a functionality of one or more of the buttons. The touch surface 646 can, for example, also be used to implement virtual or soft buttons.

In some implementations, the mobile device can present recorded audio and/or video files, such as MP3, AAC and MPEG files. In some implementations, the mobile device can include the functionality of an MP3 player. Other input/output and control devices can also be used.

Memory interface 602 can be coupled to memory 650. Memory 650 can include high-speed random access memory and/or non-volatile memory, such as one or more magnetic disk storage devices, one or more optical storage devices and/or flash memory (e.g., NAND, NOR). Memory 650 can store operating system 652, such as the iOS operating system developed by Apple Inc. of Cupertino, California. Operating system 652 may include instructions for handling basic system services and for performing hardware dependent tasks. In some implementations, operating system 652 can include a kernel (e.g., UNIX kernel).

Memory 650 may also store communication instructions 654 to facilitate communicating with one or more additional devices, one or more computers and/or one or more servers, such as, for example, instructions for implementing a software stack for wired or wireless communications with other devices. Memory 650 may include graphical user interface instructions 656 to facilitate graphic user interface processing; sensor processing instructions 658 to facilitate sensor-related processing and functions; phone instructions 660 to facilitate phone-related processes and functions; electronic messaging instructions 662 to facilitate electronic-messaging related processes and functions; web browsing instructions 664 to facilitate web browsing-related processes and functions; media processing instructions 666 to facilitate media processing-related processes and functions; GNSS/Location instructions 668 to facilitate generic GNSS and location-related processes and instructions; and HR sensor escalation instructions 670 that implement the motion activity based HR sensor escalation processes described in reference to FIGS. 1-4. Memory 650 further includes other application instructions 672 including but not limited to instructions for health monitor and/or fitness applications that use outputs of the processes described in reference to FIGS. 1-4.

Each of the above identified instructions and applications can correspond to a set of instructions for performing one or more functions described above. These instructions need not be implemented as separate software programs, procedures, or modules. Memory 650 can include additional instructions or fewer instructions. Furthermore, various functions of the mobile device may be implemented in hardware and/or in software, including in one or more signal processing and/or application specific integrated circuits.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable sub combination. Moreover, although features may be described above as acting in certain combinations and even initially claimed as such, one or more features from a claimed combination can in some cases be excised from the combination, and the claimed combination may be directed to a sub combination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

As described above, some aspects of the subject matter of this specification include gathering and use of data available from various sources to improve services a mobile device can provide to a user. The present disclosure contemplates that in some instances, this gathered data may identify a particular location or an address based on device usage. Such personal information data can include location-based data, addresses, subscriber account identifiers, or other identifying information.

The present disclosure further contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. For example, personal information from users should be collected for legitimate and reasonable uses of the entity and not shared or sold outside of those legitimate uses. Further, such collection should occur only after receiving the informed consent of the users. Additionally, such entities would take any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices.

In the case of advertisement delivery services, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services, the present technology can be configured to allow users to select to “opt in” or “opt out” of participation in the collection of personal information data during registration for services.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the content delivery services, or publicly available information.

What is claimed is:

1. A method comprising:
obtaining a window of climbing probability samples, the climbing probability samples indicating the probability that a user of the wearable device is climbing stairs;
computing a stair metric based on the window of climbing probability samples;
validating the stair metric;
obtaining a window of vertical speed samples, the vertical speed samples indicating the vertical speed of the wearable device;
determining an ascending or descending condition based on the window of vertical speed samples; and
initiating escalation of a heart rate sensor based on the validated stair metric and the ascending or descending condition.

2. A system comprising:
at least one processor;
memory storing instructions, that when executed by the at least one processor, cause the at least one processor to perform operations comprising:
obtaining a window of climbing probability samples, the climbing probability samples indicating the probability that a user of the wearable device is climbing stairs;
computing a stair metric based on the window of climbing probability samples;
validating the stair metric;
obtaining a window of vertical speed samples, the vertical speed samples indicating the vertical speed of the wearable device;
determining an ascending or descending condition based on the window of vertical speed samples; and
initiating escalation of a heart rate sensor based on the validated stair metric and the ascending or descending condition.
